(19) United States
(12) Patent Application Publication
(10) Pub. No.: US 2025/0332713 A1
(43) Pub. Date: Oct. 30, 2025

(54) ARTICULATED DEVICE

(71) Applicants: THE ASAN FOUNDATION, Seoul (KR); UNIVERSITY OF ULSAN FOUNDATION FOR INDUSTRY COOPERATION, Ulsan (KR)

(72) Inventors: Youngjin MOON, Seoul (KR); Muhammad Umer Khan NIAZI, Seoul (KR); Jaesoon CHOI, Seoul (KR); Yoon Se LEE, Seoul (KR); Usman MEHMOOD, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 19/262,356

(22) Filed: Jul. 8, 2025

Related U.S. Application Data
(63) Continuation of application No. PCT/KR2023/020900, filed on Dec. 18, 2023.

(30) Foreign Application Priority Data
Mar. 8, 2023 (KR) 10-2023-0030715

(51) Int. Cl. B25J 9/06; A61B 34/30; B25J 9/10

(52) U.S. Cl. CPC B25J 9/06; A61B 34/30; B25J 9/1045; A61B 2034/301

(57) ABSTRACT

Disclosed is an articulated device including a base member, a shuttle member disposed in the base member to be movable to any one of a first position or a second position disposed to be spaced apart from the first position on the base member, and a unit joint provided between the base member and the shuttle member to be movable in the base member together with the shuttle member, and including an elastic member that generates an elastic force to the base member and the shuttle member.

### CROSS-REFERENCE TO RELATED APPLICATIONS

The present application is a continuation of International Patent Application No. PCT/KR2023/020900, filed on Dec. 18, 2023, which is based upon and claims the benefit of priority to Korean Patent Application No. 10-2023-0030715 filed on Mar. 8, 2023. The disclosures of the above-listed applications are hereby incorporated by reference herein in their entirety.

### BACKGROUND

Embodiments of the present disclosure described herein relate to an articulated device. More specifically, the present disclosure relates to an articulated device that provides a dual-state mode, by which a locking mode for maintaining structural rigidity and an extension mode for increasing flexibility may be switched to activate different functions between multiple unit joints, and that may utilize this to perform stable surgical or procedural operations.

In general, the robotics technology and robotics industry fields are being researched and developed into industrial robots used in industrial fields and medical robots used in medical fields.

The robot includes a plurality of joint assemblies to enable joint movements that are similar to those of the joints of the human body, and wires of the robot joint wire connection structure having a plurality of joint assemblies are connected to each other, so that rotational movements of the plurality of joint assemblies may be operated by pulling the wires.

In particular, flexible robots used in the medical field are generally used for surgeries or procedures, and mobility is important in the narrow interior of the human body, and to this end, an articulated device is generally configured such that a plurality of multi-joints are connected to each other.

Accordingly, because robots in the medical field are moved in narrow spaces, it is essential for flexible robots to be miniaturized, and when moving in the bent area of the biological tissue, they have to be able to bend by rotating each multi-joint along the curve, and perform forward and backward movements along with the bending. In this case, it is important to closely control the tension of the wire and the rotation of the joint assembly when the rotational movement of the joint assembly is operated through the wire. To implement this, stretchable back bone mechanisms, origami mechanisms, and spring mechanisms in the form of flexible robots have been proposed.

However, the structure of conventional flexible robots requires a large number of actuators, is bent in an unpredictable shape when returning, and has insufficient structural rigidity under load conditions.

In addition, the structure of conventional flexible robots has a limitation that they may only be rotated at a single preset curvature in a curved area because the rotation angle between the joints has a single preset range.

### SUMMARY

Embodiments of the present disclosure provide an articulated device that may provide a dual-state mode for switching to a locking mode of maintaining a structural rigidity and an extension mode of increasing flexibility by applying a continuum robot and allow a stable surgery or procedure by utilizing this as a shuttle member that connects unit joints between one unit joint and another unit joint may be moved to two different positions and rotation angles between joints may be adjusted.

Problems to be solved by the present disclosure are not limited to the problems mentioned above, and other problems that are not mentioned will be clearly understood by those skilled in the art from the following description.

An articulated device according to an aspect of the present disclosure may include a base member, a shuttle member disposed in the base member to be movable to any one of a first position or a second position disposed to be spaced apart from the first position on the base member, and a unit joint provided between the base member and the shuttle member to be movable in the base member together with the shuttle member, and including an elastic member that generates an elastic force to the base member and the shuttle member.

The base member may include a body part formed such that at least a partial area of an inner peripheral surface thereof has a spherical surface having a specific radius of curvature, and a protrusion protruding from the body part along a lengthwise direction thereof, and including a rib protruding from an outer peripheral surface thereof along a circumferential direction thereof, and the shuttle member may be disposed in any one of the first position spaced apart from the body part with respect to the rib or the second position adjacent to the body part.

A central area of the elastic member may be formed to be spaced apart from the shuttle member and be bent toward the protrusion, and opposite ends of the elastic member may be supported by the shuttle member, the central area of the elastic member may be elastically deformed by the rib when the shuttle member is moved to the first position or the second position, and the shuttle member may be supported by the rib while a shape of the elastic member is restored when being moved to the first position or the second position.

The base member may include a left base member and a right base member formed to be assembled or disassembled to face each other with respect to an imaginary vertical cross section passing through a spherical center of the base member, and the shuttle member may include a left shuttle member and a right shuttle member formed to be assembled or disassembled to face each other with respect to an imaginary vertical cross section passing through a spherical center of the shuttle member.

A pair of elastic members may be provided, the shuttle member may include a pair of first guide parts protruding in a ring shape along inner peripheral surfaces of the left shuttle member and the right shuttle member, respectively, a pair of second guide parts having the same shapes as those of the first guide parts inside the left shuttle member and the right shuttle member, and disposed to be spaced apart from each other, a pair of first retainers disposed on a lower side of the first guide parts, respectively, a pair of second retainers disposed on an upper side of the second guide parts, respectively, and mounting grooves formed such that the pair of the elastic members are mounted to span across opposite ends of each of the first guide parts and opposite ends of each of the second guide parts, which face each other in an imaginary vertical cross section of the first shuttle member, and an outer peripheral surface of the shuttle member may be formed to have a spherical surface having the same radius of curvature as that of a spherical surface of an inner peripheral surface of the body part.

The articulated device may further include a first wire, an end of which is connected to the first retainer through a first through-hole formed in the first guide part, an opposite end of which extends from a tip end of the hollow protrusion to an interior of the body part via an inner hollow area, and that raises the shuttle member to the first position when the opposite end thereof is pulled, and a second wire, an end of which is connected to the second retainer through a second through-hole formed in the second guide part, an opposite end of which extends to an interior of the body part through a third through-hole formed in an area, in which the body part and the protrusion are adjacent to each other, and that lowers the shuttle member to the second position when the opposite end thereof is pulled.

The first retainers and the second retainers may be disposed to be spaced apart from an outer peripheral surface of the protrusion such that the rib is located in a position overlapping inner peripheral surfaces thereof in a horizontal direction, and opposite ends of each of the first retainers and the second retainers may be disposed to support side surfaces of the pair of elastic members while central areas of the elastic members interfere with the rib to be deformed.

A plurality of unit joints may be provided, and the plurality of unit joints may be connected to each other in series to form a multi-joint, and a body part of any one of a pair of unit joints connected to each other may be coupled to a shuttle member of the other one of the unit joints to be pivoted and tilted.

The unit joints may include rotation restricting members disposed to be spaced apart from each other at a preset interval along a circumferential direction of the protrusion, on the body part, and protruding along a lengthwise direction of the protrusion, and adjustment of a coupling direction or angle of the base member of an adjacent unit joint sphere-coupled to an outside of the shuttle member is relatively restricted when the shuttle member is disposed in a second position, compared to when the shuttle member is disposed in a first position.

The articulated device may further include one or motors that provide a rotational force, and a spool connected to the motor, and wind or unwind the plurality of first wires and the plurality of second wires connected to the plurality of unit joints, respectively, the spool may wind and pull the plurality of first wires and unwinds the plurality of second wires for switching into an extension mode of expanding the coupling direction or angle when the motor is rotated in one direction, and the spool may unwind the plurality of first wires and may wind and pull the plurality of second wires for switching into a fixing mode of fixing or relatively restricting the coupling direction or angle when the motor is rotated in an opposite direction.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a first unit joint of an articulated device according to an embodiment of the present disclosure;

FIG. 2 is a perspective view illustrating an inner area of a shuttle member of the present disclosure;

FIG. 3 is an exploded perspective view of an articulated device of the present disclosure;

FIGS. 4A and 4B are cross-sectional views illustrating an articulated device of the present disclosure;

FIGS. 5A and 5B are longitudinal cross-sectional views illustrating a state, in which a first unit joint and a second unit joint of the present disclosure are relatively rotated;

FIGS. 6A and 6B are front views illustrating an articulated device according to another embodiment of the present disclosure;

FIGS. 7A and 7B are longitudinal cross-sectional views illustrating a state, in which a first unit joint and a second unit joint of the present disclosure are relatively rotated; and

FIG. 8 is a reference diagram illustrating a state of driving an articulated device of the present disclosure.

### DETAILED DESCRIPTION

The same reference numerals denote the same elements throughout the present disclosure. The present disclosure does not describe all elements of embodiments. Well-known content or redundant content in which embodiments are the same as one another will be omitted in a technical field to which the present disclosure belongs. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout the specification, when we say that a part is “connected” to another part, this includes both direct connections as well as indirect connections.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

Hereinafter, an articulated device according to the present disclosure will be described with reference to the drawings.

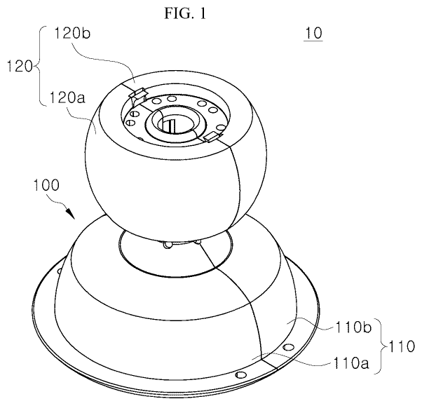

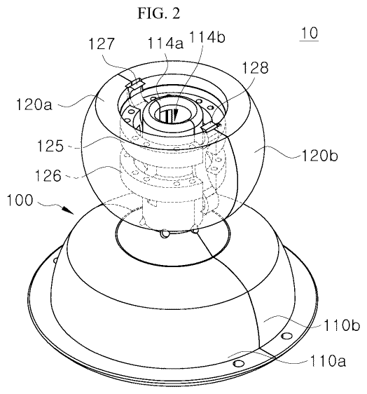

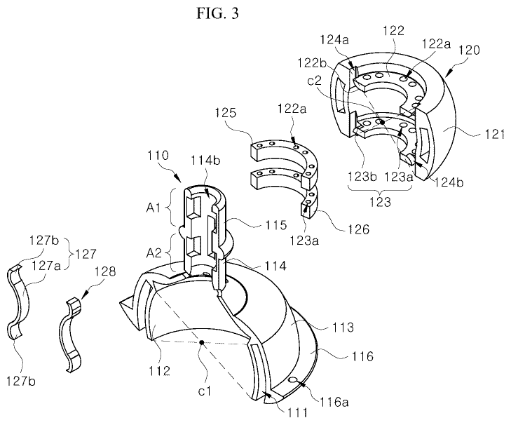

FIG. 1 is a perspective view illustrating a first unit joint of an articulated device according to an embodiment of the present disclosure, FIG. 2 is a perspective view illustrating an inner area of a shuttle member of the present disclosure, and FIG. 3 is an exploded perspective view of the articulated device of the present disclosure.

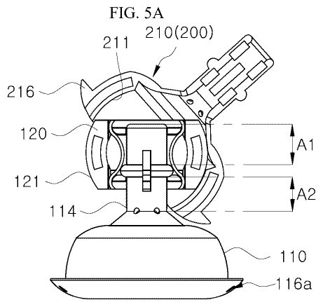

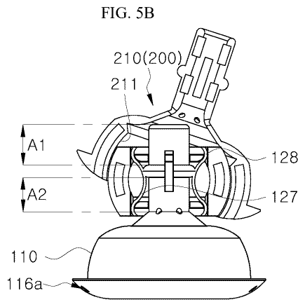

An articulated device 10 according to an embodiment of the present disclosure includes a first unit joint 100 and at least one second unit joint (see FIG. 5) 200. In FIG. 1, only the first unit joint 100 is illustrated.

The first unit joint 100 includes a first base member 110, a first shuttle member 120, and an elastic member 127 and 128.

The first base member 110 includes a left base member 110a and a right base member 110b. Here, the concepts of left and right may be changed to the concepts of upper and lower. That is, the first base member 110 includes a left base member 110a and a right base member 110b with respect to a longitudinal cross-section that passes through the center thereof, and they may be assembled or disassembled to face each other through coupling structures provided on opposite sides with respect to an imaginary vertical plane that passes through a center cl of a spherical inner peripheral surface of the first base member 110.

The first base member 110 may be configured such that the left base member 110a and the right base member 110b may be formed to be symmetrical to each other, or may be configured in a structure, in which a recess structure is provided on one side and a projection structure is provided on an opposite side such that they are fitted with and coupled to each other. In the embodiment, a structure, in which a projection part (not illustrated) is formed on the left base member 110a and a recess part 111 is formed on the right base member 110b, and they are assembled by using a coupling structure of the recess part and the projection part, will be described as an example. However, because the left base member 110a and the right base member 110b have only a difference in the coupling structure and are the same in terms of the remaining appearances and functions, the first base member 110, in which the left base member 110a and the right base member 110b are coupled to each other, will be described hereinafter. Furthermore, the same reference numerals below represent the same components.

The first base member 110 includes a first body part 113, in which at least a portion of an inner peripheral surface 112 is formed as a spherical surface, and a first protrusion 114 that protrudes long from an outside of the first body part 113.

As illustrated in FIG. 1, the first body part 113 is formed in a substantially hemispherical shape.

At least a partial area of the inner peripheral surface 112 of the first body part 113 is formed as a spherical surface having the same radius of curvature as that of an outer peripheral surface 121 of the first shuttle member 120.

The first protrusion 114 protrudes upward from an upper center of the first body part 113. The first protrusion 114 has a substantially cylindrical shape. The first protrusion 114 is provided with a rib 115 that protrudes along a circumferential direction on an outer peripheral surface thereof. One cross section of the rib 115 is formed to be substantially semicircular or elliptical, a first position A1 is formed on an upper side of the first protrusion 114 spaced apart from the first body part 113 with respect to the position of the rib 115, and a second position A2 is formed on a lower side of the first protrusion 114 that is adjacent to the first body part 113. The first body part 113 and the first protrusion 114 provide a hollow structure having an empty interior.

A tip end of an outer peripheral surface of the first body part 113 may be provided with a flange member 116 that protrudes outward.

Next, the first shuttle member 120 is formed with a spherical surface on an outer peripheral surface thereof and is coupled onto the first protrusion 114. In this case, the outer peripheral surface 121 of the first shuttle member 120 is formed with the same curvature as that of the inner peripheral surface 112 of the first body part 113 so that it may make spherical contact with the inner peripheral surface 112 of the first body part 113.

The first shuttle member 120 is formed such that a left shuttle member 120a and a right shuttle member 120b are assembled or disassembled to face each other with respect to an imaginary vertical plane that passes through the center cl of the spherical surface of the outer peripheral surface. A description will be made with reference to a state, in which the left shuttle member 120a and the right shuttle member 120b of the first shuttle member 120 are coupled to each other.

The first shuttle member 120 includes a first guide part 122, a second guide part 123, a first retainer 125, a second retainer 126, and a pair of elastic members.

Hereinafter, for convenience of description, a description will be made while the pair of elastic members include a first spring 127 and a second spring 128.

The first guide part 122 protrudes internally horizontally in a ring shape along the inner peripheral surfaces of the left shuttle member 120a and the right shuttle member 120b. Accordingly, when the left shuttle member 120a and the right shuttle member 120b are coupled to each other, the first guide part 122 is formed in a single ring shape. At least two first through-holes 122a are formed on the first guide part 122. The purposes and effects of the first through-holes 122a will be described later.

The second guide part 123 has the same shape as the first guide part 122 and is spaced apart in a downward direction. Of course, when the first shuttle member 120 is overturned by 180° while being formed symmetrically in the upward/downward and leftward/rightward directions, the positions of the first guide part 122 and the second guide part 123 may be changed in the upward/downward direction. At least two second through-holes 123a are formed on the second guide part 123. Again, the purposes and effects of the second through-holes 123a will be described later.

The first retainer 125 is disposed to be closely attached to a lower portion of the first guide part 122. Furthermore, the first retainer 125 is formed with a first through-hole 122a to be located on the same line as that of the first through-hole 122a of the first guide part 122.

The second retainer 126 is disposed to be closely attached to an upper portion of the second guide part 123. Furthermore, the second retainer 126 is formed with a second through-hole 123a to be located on the same line as that of the second through-hole 123a of the second guide part 123.

Furthermore, as illustrated in FIG. 3, in a description of the right shuttle member 120b, mounting grooves 124a and 124b are formed between opposite ends of the first guide part 122 and the second guide part 123 and the inner peripheral surface of the right shuttle member 120b. Of course, although not illustrated in the drawings, mounting grooves (not illustrated) having the same shape and size as those described above are also formed in the left shuttle member 120a. The mounting grooves 124a and 124b are formed at opposite ends of each guide part 122 and 123, respectively, and a first spring 127 is installed in one mounting groove 124a and a second spring 128 is installed in the other mounting groove 124b.

The first spring 127 and the second spring 128 are formed in the same shape and are positioned to face each other in an interior of the first shuttle member 120. The first spring 127 and the second spring 128 are provided between the first base member 110 and the first shuttle member 120 so as to be movable to the first base member 110 together with the first shuttle member 120, and generate an elastic force in the first base member 110 and the first shuttle member 120.

Hereinafter, the shape and the coupling structure of the first spring 127 will be described in detail, and a repeated description of the second spring 128 will be omitted.

The first spring 127 may have a height corresponding to the height of the first shuttle member 120, and may be formed into an overall streamlined, vertically symmetrical shape.

A central area of the first spring 127 is spaced apart from the first shuttle member 120 to be bent to face an outer circumference of the first protrusion 114, and both ends of the first spring 127 are supported by the mounting groove 124a of the first shuttle member 120.

Accordingly, when the first spring 127 is inserted into the mounting groove 124a, a central area 127a protrudes convexly toward the first protrusion 114, and opposite ends 127b are formed in a ring or hook shape to be closely attached to a bottom surface 122b of the first guide part 122 and an upper surface 123b of the second guide part 123, respectively.

Due to the configuration, the first shuttle member 120 is coupled to be slidable on the first protrusion 114, and due to the elastic deformation of the first spring 127 and the second spring 128, it may be moved to any one of the first position A1 or the second position A2 formed on the first protrusion 114 while the shape thereof is restored after interfering with the rib 115.

When the first shuttle member 120 is moves to the first position A1 or the second position A2, the central areas of the first spring 127 and the second spring 128 are elastically deformed by the rib 115, and when the first shuttle member 120 is located at the first position A1 or the second position A2, the first spring 127 and the second spring 128 are supported by the rib 115 while restoring the shapes thereof.

In this way, in the process of the first spring 127 and the second spring 128 being elastically deformed, the first retainer 125 and the second retainer 126 are disposed to support the side surfaces of the springs 127 and 128, respectively. Of course, because the opposite ends 127b of the first spring 127 and the second spring 128 are supported by the tip ends of the first guide part 122 and the second guide part 123 in the interiors of the mounting groove 124a and 124b, respectively, the mounting groove 124a and 124b may be prevented from be moved in the upward/downward direction, and because the side surfaces of the mounting grooves 124a and 124b and the opposite ends of the first retainer 125 and the second retainer 126 support the side surface of a central portion 127a, the central portion 127a may be prevented from being moved in the leftward/rightward direction.

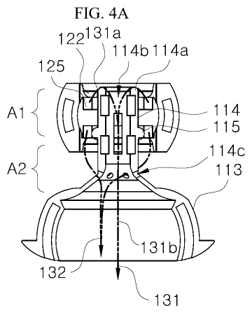

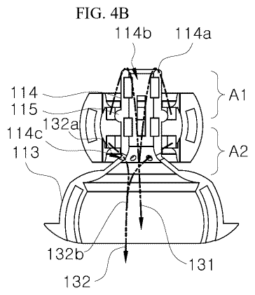

FIGS. 4A and 4B illustrate a state, in which the first shuttle member 120 is slid on the first base member 110 through the first wire and the second wire.

Here, FIG. 4A illustrates a state, in which the first shuttle member 120 is raised to the first position A1 as a first wire 131 is pulled.

Referring to FIG. 4A, one end 131a of the first wire 131 is connected to the first retainer 125 through the first through-hole 122a formed in the first retainer 125 and the first guide part 122, and an opposite end 131b thereof extends into the interior of the first body part 113 while being introduced into the hollow interior through a tip end 114a of the first protrusion 114.

In this case, a height of an uppermost end of the first guide part 122 is disposed lower than a height of an uppermost end of the tip end 114a of the first protrusion 114. Accordingly, when an opposite end of the first wire 131 is pulled, the first wire 131 pulls the first shuttle member 120 to the first position A1 while the direction thereof is changed at the upper tip end 114a of the first protrusion 114. Furthermore, the first wire 131 may be pulled by using a single wire, but to be safer and reduce friction, the wires may be connected in positions that face each other, and may be pulled to pass through a center 114b of the hollow area of the first protrusion 114.

Furthermore, FIG. 4B illustrates a state, in which the first shuttle member 120 is lowered to the second position A2 as a second wire 132 is pulled.

Referring to FIG. 4B, one end 132a of the second wire 132 may be connected to the second retainer 126 through the second through-hole 123a formed in the second guide part 123, and an opposite end 132b thereof may be pulled to a lower side of the first body part 113 while the first protrusion 114 and the first body part 113 are introduced into the interior of the hollow first body part 113 through a third through-hole 114c formed in an adjacent area.

In this case, the heights of the second guide part 123 and the second retainer 126 are located at a site that is higher than the height of the third through-hole 114c, and when the second wire 132 is pulled, a tensile force of the first wire 131 is released.

Accordingly, in the articulated device 10 according to an embodiment of the present disclosure, the first shuttle member 120 is disposed on the first base member 110 to be slid to any one of the first position A1 or the second position A2.

Due to the configuration, a process of operating the articulated device according to an embodiment of the present disclosure will be described below by using FIGS. 5A to 7B.

FIGS. 5A and 5B are longitudinal cross-sectional views illustrating an example of an articulated device of the present disclosure, in which a first unit joint 100 and a second unit joint 200 are connected to each other in series, and the second unit joint 200 is rotated relative to the first unit joint 100.

Referring to FIGS. 5A and 5B, the second unit joint 200 having the same structure as that of the first unit joint 100 is coupled to the first unit joint 100. In FIGS. 5A and 5B, only a second base member 210 of the second unit joint 200 is illustrated.

An inner peripheral surface 211 of the second base member 210 is coupled to an area of the outer peripheral surface 121 of the first shuttle member 120 to make spherical contact, so that the second base member 210 is disposed to be pivoted and tilted on the first shuttle member 120.

FIG. 5A illustrates a state, in which the second base member 210 is rotated while the first shuttle member 120 is located in the first position A1 of the first protrusion 114. In FIG. 5A, the second base member 210 is switched to an extension mode, in which it may be rotated at a maximum angle on the first shuttle member 120. Although not illustrated in the drawings, the second base member 210 may be rotated to be bent in a direction, in which another wire (not illustrated) that connects a through-hole 116a formed on a flange member 116 of the first base member 110 and a through-hole (not illustrated) formed on a flange member 216 of the second base member 210.

FIG. 5B illustrates a state, in which the second base member 210 is rotated while the first shuttle member 120 is located in the second position A2 of the first protrusion 114. In FIG. 5B, the second base member 210 is switched to a fixing mode, in which it may be rotated at a relatively small angle on the first shuttle member 120. In the state of FIG. 5B, a rotation angle of the second base member 210 becomes relatively small because the first body part 113 is located to interfere with the second base member 210 in a direction, in which the second base member 210 is rotated.

Accordingly, when the first shuttle member 120 is located in the first position A1 of the first protrusion 114, the coupling direction or rotation angle of the second base member 210 may be adjusted more significantly than when it is located in the second position A2, and when the first shuttle member 120 is located in the second position A2, the coupling direction or rotation angle of the second base member 210 may be adjusted less while a higher structural rigidity is maintained.

Furthermore, the second base member 210 may be rotated around the first shuttle member 120 in various directions. For example, the rotational direction of the second base member 210 may be adjusted in various ways with respect to imaginary axes in various directions that pass through the spherical center cl of the outer peripheral surface of the first shuttle member 120.

Furthermore, although not illustrated in the drawings, a coating layer (not illustrated) may be further provided on any one or opposite sides of the outer peripheral surface of the shuttle member of any one of the pair of unit joints that are connected to each other and the inner peripheral surface of the base member 210 of the other unit joint. For example, the coating layer may be formed through ceramic coating. Because the ceramic coating may reduce frictional resistance while providing corrosion resistance, heat resistance, electrical insulation, airtightness, and the like, and, it may provide a lubricating function between the outer peripheral surface of the shuttle member of any one unit joint that is spherically coupled and the inner peripheral surface of the base member of the other unit joint. Of course, the coating layer may be provided in all of the areas, in which the unit joints make spherical contact. In addition, solid lubricating coating, film coating, and the like that provide a lubricating function may be applied to the coating layer.

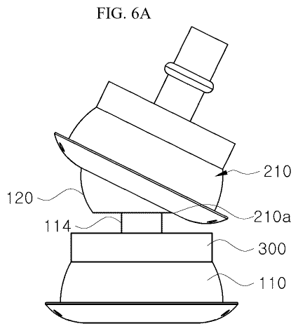

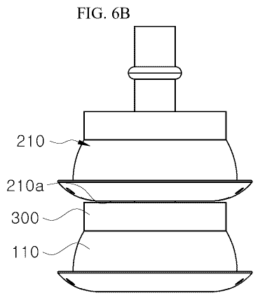

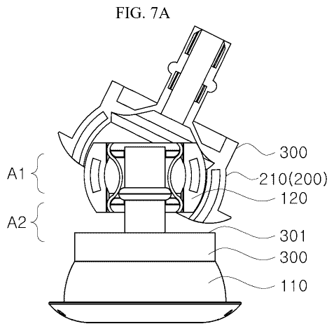

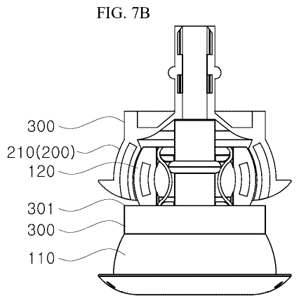

FIGS. 6A and 6B are front views illustrating an articulated device according to another embodiment of the present disclosure, and FIGS. 7A and 7B are longitudinal cross-sectional views illustrating a state, in which a first unit joint and a second unit joint of the present disclosure are rotated with respect to each other. Referring to FIGS. 6A to 7B, an articulated device 20 according to another embodiment of the present disclosure is provided with a rotation restricting member 300 in addition to the articulated device 10 according to the above-described embodiment.

The rotation restricting member 300 is disposed on the first base member 110 to be spaced apart at a preset interval along the circumferential direction of the first protrusion 114, and protrudes along the direction, in which the first protrusion 114 protrudes.

The rotation restricting member 300 has a cylindrical shape having a substantially low height, and an upper surface 301 thereof may be formed as a plane.

FIG. 7A is in the same context as the above-described extension mode. The first shuttle member 120 is disposed in the first position A1, and the second base member 210 is sphere-coupled to the outer peripheral surface of the first shuttle member 120. Accordingly, the second base member 210 may be rotated and tilted while making spherical contact with the first shuttle member 120 therearound, and in the extension mode, the second base member 210 may be rotated at the maximum coupling direction or angle.

FIG. 7B is in the same context as the above-described fixing mode. However, because a cylindrical rotation restricting member 300 is provided on the first base member 110, the second base member 210 sphere-coupled to the outer peripheral surface of the first shuttle member 120 disposed in the second position A2 is restricted so that the coupling direction or rotation angle cannot be changed. In the fixing mode of FIG. 7B, because the upper surface 301 of the rotation restricting member provided on the first base member 110 and a bottom surface 210a or the lower tip end of the second base member 210 are disposed to almost contact each other, a gap required for the rotation of the second base member 210 may be restricted within approximately an assembly tolerance level.

Accordingly, the articulated device 20 according to another embodiment of the present disclosure may more reliably restrain the rotation of the second base member 210 in the fixing mode compared to the articulated device 10 according to an embodiment, and thus may significantly increase the structural rigidity in the fixing mode.

Of course, although not illustrated in the drawing, the design may be changed to provide a coupling direction or rotation angle of the second base member 210 within a specific range by adjusting the height of the rotation restricting member 300 relatively lower in the fixing mode of FIG. 7B. In this case, because the coupling direction or rotation angle of the second base member 210 may be provided even in the fixing mode, bending may be coped with more effectively.

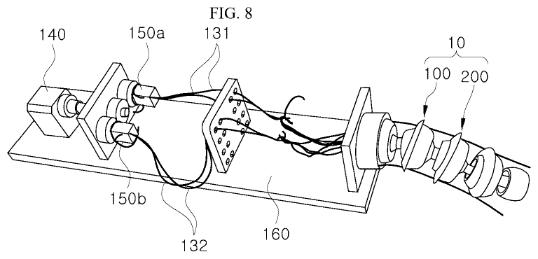

FIG. 8 is a reference diagram illustrating a state of driving an articulated device of the present disclosure.

Referring to FIG. 8, the articulated device 100 and 200 includes a driving member including a motor 140 and a spool 150a and 150b. The driving member may be provided on one installation plate 160 together with the above-described articulated device.

At least one or a plurality of motors 140 are provided, and provide a driving force to each spool 150 through rotation thereof.

The spools 150a and 150b receive a driving force from the motor 140 and is rotated in one direction (e.g., a forward direction) or an opposite direction (e.g., a reverse direction) to wind or unwind the plurality of first wires 131 or the plurality of second wires 132 connected to a plurality of unit joints.

For example, when the motor 140 is rotated in one direction, the first spool 150a may wind and pull a plurality of first wires 131 and the second spool 150b may unwind a plurality of second wires 132 for switching to the extension mode, and conversely, when the motor 140 is rotated in an opposite direction, the first spool 150a may unwind the plurality of first wires 131 and the second spool 150b may wind and pull the plurality of second wires 132 for switching to the fixing mode.

Although not illustrated in the drawing, this driving member may be configured not only for switching between the extension mode and fixing mode between the first unit joint 100 and the second unit joint 200, but also to adjust the degree and direction of bending by having another wire (not illustrated).

Accordingly, according to the articulated device of the present disclosure, the unit joints may move the shuttle member from the base member to the first position or the second position, the movement function may provide an effect selectively switching the mode to the extension mode and the fixing mode while being coupled to the adjacent unit joints connected to each other, the mode between the unit joints may be switched when any one wire is pulled while the first wire and the second wire are connected to the first retainer and the second retainer, the rotation restricting member is provided in at least one unit joint to restrict the base member of another unit joint in the fixing mode from being rotated, rotation may be flexibly made to cope with various bending in the extension mode through the above-described functions, or the structural rigidity may be properly maintained in the fixing mode, in which no rotation is required.

Accordingly, according to the above-described articulated device of the present disclosure, first, the unit joints may move the shuttle member from the base member to the first position or the second position, second, the movement function may provide an effect selectively switching the mode to the extension mode and the fixing mode while being coupled to the adjacent unit joints connected to each other, third, the mode between the unit joints may be switched when any one wire is pulled while the first wire and the second wire are connected to the first retainer and the second retainer, fourth, the rotation restricting member is provided in at least one unit joint to restrict the base member of another unit joint in the fixing mode from being rotated, and fifth, rotation may be flexibly made to cope with various curving in the extension mode through the above-described functions, or the structural rigidity may be properly maintained in the fixing mode, in which no rotation is required.

Effects of the present disclosure are not limited to the effects mentioned above, and other effects not mentioned will be clearly understood by those skilled in the art from the following description.

Although the present disclosure has been illustrated and described above as a specific embodiment to illustrate the technical idea of the present disclosure, the present disclosure is not limited to the same configuration and operation as the specific embodiment as described above, and various modifications may be implemented within the scope of the present disclosure. Accordingly, such modifications should also be considered to fall within the scope of the present disclosure, and the scope of the present disclosure should be determined by the claims set forth below.

What is claimed is:

1. An articulated device comprising:
a base member;
a shuttle member disposed in the base member to be movable to any one of a first position or a second position disposed to be spaced apart from the first position on the base member; and
a unit joint provided between the base member and the shuttle member to be movable in the base member together with the shuttle member, and including an elastic member configured to generate an elastic force to the base member and the shuttle member.

2. The articulated device of claim 1, wherein the base member includes:
a body part formed such that at least a partial area of an inner peripheral surface thereof has a spherical surface having a specific radius of curvature; and
a protrusion protruding from the body part along a lengthwise direction thereof, and including a rib protruding from an outer peripheral surface thereof along a circumferential direction thereof.

3. The articulated device of claim 2, wherein the shuttle member is disposed in any one of the first position spaced apart from the body part with respect to the rib or the second position adjacent to the body part.

4. The articulated device of claim 3, wherein a central area of the elastic member is formed to be spaced apart from the shuttle member and be bent toward the protrusion, and opposite ends of the elastic member are supported by the shuttle member,
wherein the central area of the elastic member is elastically deformed by the rib when the shuttle member is moved to the first position or the second position, and
wherein the shuttle member is supported by the rib while a shape of the elastic member is restored when being moved to the first position or the second position.

5. The articulated device of claim 4, wherein the base member includes a left base member and a right base member formed to be assembled or disassembled to face each other with respect to an imaginary vertical cross section passing through a spherical center of the base member, and
wherein the shuttle member includes a left shuttle member and a right shuttle member formed to be assembled or disassembled to face each other with respect to an imaginary vertical cross section passing through a spherical center of the shuttle member.

6. The articulated device of claim 5, wherein a pair of elastic members are provided,
wherein the shuttle member includes:
a pair of first guide parts protruding in a ring shape along inner peripheral surfaces of the left shuttle member and the right shuttle member, respectively;
a pair of second guide parts having the same shapes as those of the first guide parts inside the left shuttle member and the right shuttle member, and disposed to be spaced apart from each other;
a pair of first retainers disposed on a lower side of the first guide parts, respectively;
a pair of second retainers disposed on an upper side of the second guide parts, respectively; and
mounting grooves formed such that the pair of the elastic members are mounted to span across opposite ends of each of the first guide parts and opposite ends of each of the second guide parts, which face each other in an imaginary vertical cross section of the first shuttle member, and
wherein an outer peripheral surface of the shuttle member is formed to have a spherical surface having the same radius of curvature as that of a spherical surface of an inner peripheral surface of the body part.

7. The articulated device of claim 6, further comprising:
a first wire, an end of which is connected to the first retainer through a first through-hole formed in the first guide part, an opposite end of which extends from a tip end of the hollow protrusion to an interior of the body part via an inner hollow area, and configured to raise the shuttle member to the first position when the opposite end thereof is pulled; and
a second wire, an end of which is connected to the second retainer through a second through-hole formed in the second guide part, an opposite end of which extends to an interior of the body part through a third through-hole formed in an area, in which the body part and the protrusion are adjacent to each other, and configured to lower the shuttle member to the second position when the opposite end thereof is pulled.

8. The articulated device of claim 5, wherein the first retainers and the second retainers are disposed to be spaced apart from an outer peripheral surface of the protrusion such that the rib is located in a position overlapping inner peripheral surfaces thereof in a horizontal direction, and
wherein opposite ends of each of the first retainers and the second retainers are disposed to support side surfaces of the pair of elastic members while central areas of the elastic members interfere with the rib to be deformed.

9. The articulated device of claim 7, wherein a plurality of unit joints are provided, and the plurality of unit joints are connected to each other in series to form a multi-joint, and
wherein a body part of any one of a pair of unit joints connected to each other is coupled to a shuttle member of the other one of the unit joints to be pivoted and tilted.

10. The articulated device of claim 9, wherein the unit joints include:
rotation restricting members disposed to be spaced apart from each other at a preset interval along a circumferential direction of the protrusion, on the body part, and protruding along a lengthwise direction of the protrusion, and
wherein adjustment of a coupling direction or angle of the base member of an adjacent unit joint sphere-coupled to an outside of the shuttle member is relatively restricted when the shuttle member is disposed in a second position, compared to when the shuttle member is disposed in a first position.

11. The articulated device of claim 9, further comprising:
one or motors configured to provide a rotational force; and
a spool connected to the motor, and wind or unwind the plurality of first wires and the plurality of second wires connected to the plurality of unit joints, respectively,
wherein the spool winds and pulls the plurality of first wires and unwinds the plurality of second wires for switching into an extension mode of expanding the coupling direction or angle when the motor is rotated in one direction, and
wherein the spool unwinds the plurality of first wires and winds and pulls the plurality of second wires for switching into a fixing mode of fixing or relatively restricting the coupling direction or angle when the motor is rotated in an opposite direction.

12. The articulated device of claim 10, further comprising:
one or motors configured to provide a rotational force; and
a spool connected to the motor, and wind or unwind the plurality of first wires and the plurality of second wires connected to the plurality of unit joints, respectively,
wherein the spool winds and pulls the plurality of first wires and unwinds the plurality of second wires for switching into an extension mode of expanding the coupling direction or angle when the motor is rotated in one direction, and
wherein the spool unwinds the plurality of first wires and winds and pulls the plurality of second wires for switching into a fixing mode of fixing or relatively restricting the coupling direction or angle when the motor is rotated in an opposite direction.
